(19) United States
(12) Patent Application Publication
(10) Pub. No.: US 2015/0359431 A1
(43) Pub. Date: Dec. 17, 2015

(54) BRAND-SELF PERCEPTUAL NEURAL MODEL UTILIZING THE SUPERIOR TEMPORAL SULCUS

(71) Applicants: Tomer Bakalash, Shdema (IL); Reuven Bakalash, Shdema (IL)

(72) Inventors: Tomer Bakalash, Shdema (IL); Reuven Bakalash, Shdema (IL)

(21) Appl. No.: 14/603,491

(22) Filed: Jan. 23, 2015

Related U.S. Application Data
(63) Continuation-in-part of application No. 14/304,986, filed on Jun. 16, 2014, now abandoned.
Application No. 14/304,986 is a continuation-in-part of application No. 13/636,969, filed on Dec. 26, 2012, now abandoned.
(60) Provisional application No. 61/327,711, filed on Apr. 25, 2010.

(51) Int. Cl. A61B 5/00; A61B 5/055; G01R 33/56; G06Q 30/02; G01R 33/48

(52) U.S. Cl. CPC A61B 5/0042; G06Q 30/0242; G01R 33/4806; G01R 33/5608; A61B 5/055; A61B 2503/12

(57) ABSTRACT

Brand-self perceptual neural model (BSPN) is a method for measuring marketing communication effectiveness. The model is based on neural activity indications from the Superior Temporal Sulcus (STS) and from related neural connectivity networks in the human brain, as well as neural activation patterns such as gender, motivations, emotions, cognitive load capacity, and other psychographic variables.

### CROSS-REFERENCE TO RELATED CASES

The present application claims priority based on U.S. Provisional Application No. 61/327,711 filed Apr. 25, 2010 entitled “Use of Superior Temporal Sulcus Activation as a Predictor for Emotional Engagement and Subsequent Memorability”, and on International (PCT) Patent Application No. PCT/IL2011/000326 filed Apr. 17, 2011 entitled “Method and System for Determining Memorability of Audio and/or Visual Content”, and is a Continuation In Part of U.S. application Ser. No. 13/636,969 filed Sep. 24, 2012 entitled “Method and System for Determining Potential Memorability of Audio and/or Visual Content”, and is a Continuation In Part of U.S. application Ser. No. 14/304,986 filed Jun. 16, 2014 entitled “Use of Neural Activation in the Superior Temporal Sulcus as a Predictor For Memorability of Audio and/or Visual Content and Emotional Engagement,” all of which are hereby incorporated by reference.

### FIELD OF THE INVENTION

This invention relates to methods and systems for assessing the effects of marketing audio and/or visual content.

### BACKGROUND OF THE INVENTION

Marketing effectiveness is the quality of how marketers go to market with the goal of optimizing their spending to achieve good results for both the short-term and long-term. It is also related to Marketing ROI and Return on Marketing Investment (ROMI). Marketing effectiveness is quintessential to marketing, going so far as to say it's not marketing if it's not measured.

Establishing metrics for marketing communications efforts is a challenge. It's a hot topic and one that marketing and communications professionals often spend a lot of time and energy discussing or debating. With so many choices and options available to reach your audience, it helps to not only have reliable analysis of strategies and tactical approaches going in, but to also track and measure the effectiveness of those efforts along the way. Results are really what matter. In their book “What Sticks” Rex Briggs and Greg Stuart calculated that marketers waste 37% of their marketing investment. Reasons for the waste include failure to understand underlying customer motivations for buying, ineffective messages and inefficient media mix investment (pages 19-20).

Marketing communications aren't an exact science. They should, however, lineup and provide decision-makers with evidence that demonstrates proof of performance and enables them to make sound decisions. One of the big benefits of consistently and constantly measuring your efforts is that you can make changes and correct as you go. However, tracking along the way and making correction needs a reliable measure of the marketing communication effectiveness.

Therefore, there is great need for a generic model of marketing communication that covers understanding of customer motivations and a method and means for a reliable measure of the marketing communication effectiveness.

The preferred measure embodiment of the present invention is based on Superior Temporal Sulcus (STS), which activation indicates on a positive association between social cognitive processing in exposure to ads and ad memorability.

The following prior art publications are considered to be relevant for an understanding of the novelty and importance of the use of STS activation for a reliable measure of the marketing communication effectiveness.
Aaker, Jennifer L. (1997), “Dimensions of Brand Personality,” Journal of Marketing.
Allison, Truett, Aina Puce, and Gregory McCarthy (2000), “Social Perception from Visual Cues: Role of the STS Region,” Trends in Cognitive Sciences, 1 (July), 267-78.
Canli, Turhan, Zuo Zhao, James Brewer, John Gabrieli, and Larry Cahill (2000), “Event-Related Activation in the Human Amygdala Associates with Later Memory for Individual Emotional Experience,” Journal of Neuroscience, 20 (October), RC99.
Hamann, Stephan (2001), “Cognitive and Neural Mechanisms of Emotional Memory,” Trends in Cognitive Science, (September), 394-400.
Ioannides, Andreas A., Lichan Liu, DionyssiosTheofilou, Jürgen Dammers, Tom Burne, Tim Ambler, and Steven Rose (2000), “Real Time Processing of Affective and Cognitive Stimuli in the Human Brain Extracted from MEG Signals,” Brain Topography, 13 (September), 11-19.
Kenning, Peter, HilkePlassmann, H. Kugel, W. Schwindt, A. Pieper, Michael Deppe (2007), “Neural Correlates of Attractive Ads, FOCUS-Jahrbuch 2007, Schwerpunkt: Neuroeconomy, Neuromarketing, Neuromarktforschung, Wolfgang. J. Koschnick, ed., FOCUS MagazinVerlag: Munich; 287-98.
eds., New York: Guilford Press, 601-17.
Kriegstein, Katharina V., and Anne-Lise Giraud (2004), “Distinct Functional Substrates Along the Right Superior Temporal Sulcus for the Processing of Voices,” NeuroImage, 22 (June), 948-55.
Leibenluftet. Al. (2004). “Mother's Neural Activation in Response to Pictures of Their Children and Other Children”, Biological Psychiatry, vol. 56, no. 4, 225-232 U.S. Pat. No. 6,415,048 B1 (SCHNEIDER MICHAEL BRET [US]) 2 Jul. 2002.
WO 2009/111652 A1 (UNIV NEW YORK [US]; HASSON URI [US]; MALACH RAFT [IL]; HEEGER DAVID [US]) 11 Sep. 2009.
Grossman, E., M. Donnelly, R. Price, D. Pickens, V. Morgan, G. Neighbor, and R. Blake. (2000); “Brain Areas Involved in Perception of Biological Motion,”Journal of Cognitive Neuroscience, 12. (5), 711-20.
Binder, Jeffey. R., Julie. A. Frost, Thomas. A. Hammeke, Patrick. S. F. Bellgowan, Jane. A. Springer, Jacqueline. N. Kaufman, and Edward. T. Possing (2000), “Human Temporal Lobe Activation by Speech and Nonspeech Sounds,” Cerebral Cortex 10 (5), 512-28.
Rimol, Lars M., KarstenSpecht, Susanne Weis, Robert Savoy, and Kenneth Hugdahl. (2005), “Processing of Sub-syllabic Speech Units in the Posterior Temporal Lobe: An fMRI Study,” NeuroImage 26 (4), 1059-67.
Uppenkamp, Stefan, Ingrid S. Johnsrude, Dennis Norris, William Marslen-Wilson, and Roy D. Patterson. (2006), “Locating the Initial Stages of Speech, Sound Processing in Human Temporal Cortex,” Neuroimage, 31 (3), 1284-96.
Hoffman, Elizabeth A., and James V. Haxby (2000), “Distinct Representations of Eye Gaze and Identity in the Distributed Human Neural System for Face Perception,” Nature Neuroscience, 3, 80-4.
Ishai, Alumit, Conny F. Schmidt, and Peter Boesiger (2005), “Face Perception is Mediated by a Distributed Cortical Network,” Brain Research Bulletin, 67 (1-2), 87-93.
Kenning et. al. (2007) “Applications of Functional Magnetic Resonance Imaging for Market Research”, Qualitative Market Research, Vol. 10, no 2, 135-152.
U.S. Pat. No. 6,099,319 to Zaltman.
US Patent Publication No. 2009/030303 to Pradeep.

Consumers are exposed to a considerable number of advertisements on a daily basis. Often there is a significant time delay between exposure to an ad and the decision making of consumers concerning the product. Thus, in order for an ad to be effective, it is crucial that it be memorable. Recognizing the importance of ad memorability, many researchers have attempted to determine factors that enhance memory of an ad. One such factor is affective response following exposure to anad. Further, we show that this affective response is connected to social-cognitve processing that at a time effects the self-preceptual psychological processing.

One difficulty in the study of the process by which affect influences memory relates to the measures that are indicative of the underlying process. Thus far, researchers have used a variety of measures to assess emotional reactions, including verbal self-report, visual self-report, moment-to-moment ratings, and autonomic measures. These measures, however, are usually limited in their ability to provide insights regarding the mechanism by which affect influences memory.

Functional neuroimaging and other methods have indicated that emotional stimuli engage specific neural mechanisms that enhance memory. Studies have pointed to the key role of the amygdala, an almond-shaped region of the medial temporal lobe, in enhancing memory for emotional stimuli (Hamann 2001). Research has shown a high degree of correlation between amygdala activation during encoding and subsequent memory for emotional experiences (Canli et al. 2000) and that the memory-enhancing effects of emotional arousal involve interactions between sub-cortical and cortical structures coordinated by the amygdala.

Ioannides et al. (2000) employed MEG (Magnetoencephalography) brain imaging in order to observe the differences in brain activation during exposure to affective and cognitive advertising stimuli. Their results revealed significant differences in brain activation between affective and cognitive advertising segments. Their data suggest that cognitive advertisements produce stronger activity in the posterior parietal areas and the superior prefrontal cortex than affective ads. In addition, they found that affective advertisements modulated activity in the orbitofrontal and retrosplenial cortices, the amygdala, and the brainstem.

When neural cells are active they increase their consumption of energy from glucose and switch to less energetically effective, but more rapid anaerobic glycolysis. The local response to this energy utilization is to increase blood flow to regions of increased neural activity. This leads to local changes in the relative concentration of oxyhemoglobin and deoxyhemoglobin and changes in local cerebral blood volume and in local cerebral blood flow. Functional magnetic imaging (fMRI) measures this hemodynamic response (the change in blood flow) related to neural activity in the brain or spinal cord using blood oxygenation level dependent (BOLD) contrast. A study by Kenning et al. (2007) using fMRI to monitor neural activity, indicated that the attractiveness of 30 pre-selected and classified print advertisements was correlated with changes in brain activity in the nucleus accumbens, the posterior cingulate, the medial prefrontal cortex, higher-order visual cortices, and the fusiform face area.

U.S. Pat. No. 6,099,319 to Zaltman discloses exposing a subject to advertising. Brain responses to the advertising are measured from neuroimaging data. The results of the measurements are used to predict future behavior of the subject with respect to purchase or consumption of products. However, Zaltman does not mention superior temporal sulcus (STS), or any link between STS, memorability, and future behavior of the subject.

US Patent Publication No. 2009030303 discloses use of neuro-response data to evaluate the effectiveness of stimulus materials such as marketing and entertainment materials. A data collection mechanism, including multiple modalities such as, electroencephalography (EEG), functional magnetic resonance imaging (fMRI), electrooculography (EOG), galvanic skin response (GSR), collects response data from subjects exposed to marketing and entertainment stimuli.

Some aspects of the present invention are based on the novel observation that exposure to memorable audio and/or visual content causes neural stimulation of the STS. All of the prior art suggests that the STS is neither associated with memorability nor with emotional-memory. A state-of-the-art meta-analytical study was published in Neuropsychologia by Murty, Vishnu P., Maureen Ritchey, Alison R. Atcock, and Kevin S. LaBar, titled “fMRI Studies of Successful Emotional Memory Encoding: A Quantitative Meta-Analysis” (Neuropsychologia, 48, 3459-69). This paper was published on July 2010 (three month after the priority date of our U.S. Provisional Application No. 61/327,711 filed Apr. 25, 2010). The authors presented an extensive meta-analysis of memory encoding for emotional versus neutral information, employing a meta-analytical approach. The meta-analysis revealed consistent clusters within bilateral amygdala, anterior hippocampus, anterior and posterior parahippocampalgyrus, the ventral visual stream, left lateral prefrontal cortex and right ventral parietal cortex. However, the STS is not mentioned in this state-of-the-art meta-analytical study, as the STS was not identified for its role in emotional memory.

The fact that memorable ads (as opposed to unmemorable ads) produce significantly higher neural activation in the STS is completely novel. Past research did not identify this area as involved in memory processes; nor in emotional processes that lead to memorability (see Murty et al. 2010 and FIG. 3).

Activation of the STS has been associated with a variety of functions, including biological motion (e.g., Grossman et al. 2000), processing of speech (e.g., Binder et al. 2000; Rimol et al. 2005; Uppenkamp et al. 2006), eye gazing (e.g., Hoffman and Haxby 2000), and facial perception (e.g., Ishai, Schmidt, and Boesiger 2005), none of which is associated with memory or emotional-memory.

Furthermore, the innovative evidence we found of excessive STS neural activation, and heightened self-reported emotional reaction in exposure to the memorable ads, suggests links among ad-elicited emotional arousal and ad memorability that can be measured by neural means in the STS.

Distinct activation in STS may shed light on the process involved in emotional memory in our case. The STS is a cortical structure located in the temporal lobe of the brain. Activation of the STS has been associated with a variety of functions, including biological motion (e.g. Grossman et al., 2000), processing of speech (e.g. Binder et al., 2000; Rimol et al., 2005; Uppenkamp et al., 2006), eye gazing (e.g., Hoffman and Haxby 2000), and facial perception (e.g., Ishai, Schmidt, and Boesiger 2005). More recently, Hein and Knight (2008) suggested that the function of the STS is determined by the co-activation with other brain regions. Consistent with research suggesting that the STS is associated with social cognitive processes (Allison, Puce, and McCarthy 2000; Vander Wyk et al. 2009), may suggests that emotional processing involves social cognitive processes whereby individuals identify others' emotions and appraise these emotions to affect one's own emotional state. The indication of higher STS activation in memorable ads suggests a positive association between social cognitive processing in exposure to ads and ad memorability. Furthermore, evidence of heightened emotional reaction in exposure to the memorable ads, as indicated in the amygdala activation, suggests links among ad-elicited emotional arousal, socio-cognitive processing, and ad memorability.

Thus, based on our findings, there is a link between emotional reaction to ads, self-perceptual processes and social-cognitive processing.

### SUMMARY OF THE INVENTION

Brand-self perceptual neural model (BSPN) FIG. 1b establishes a method for measuring marketing communication effectiveness. The method is based on neural activity indications from the Superior Temporal Sulcus (STS) and related neural connectivity networks, in the human brain.

Aspects of our current invention establish a model to evaluate the effectiveness of marketing communication, based on neural measuring of communication-elicited emotional arousal, and of social cognitive processes in the relationship with marketing communication.

The underlying research to the present invention has discovered a link between ad memorability and excessive neural activation of the superior temporal sulcus (STS). The superior temporal sulcus (STS) is a brain structure indicative of social perceptual and social cognitive processes. Its activation can be measured by neural means. The proposed methodological model measures the neural activation levels and the neural patterns of the STS and related neural connectivity networks, calculates the differential effects the described above variables have upon the STS neural activation patterns (gender, motivations, emotions, cognitive load capacity, and other psychographic variables) to determine marketing communication's potential effectiveness.

The measured neural activation is indicative of social cognition and emotions of an exposed individual to marketing material. Some or all of the differential variables are weighted with the measured STS score. Emotional valence is one of the differential variables having a binary value, which can invert the meaning of the individual STS score, e.g. from positive to negative attitude toward the specific marketing material. Emotional arousal, another differential variable, having high and low values, may indicate on a temporal emotional state of the individual induced by the marketing communication stimuli. These two parameters must be taken into account when processing the STS score (and related neural connectivity networks) for the final marketing communication effectiveness (MCE). Another differential parameter is the individual's gender, which might be relevant in some marketing communications, but irrelevant in others. The individual's parameters of emotional valence, emotional arousal and gender, which are externally accessible, as well as other differential variables according to needs, must be weighted with the STS score to normalize the final MCE result across multiple individuals. Finally, the marketing communication effectiveness (MCE) is given by the following equation:

MCE=STS_{activation}_{_{—}}ₗₑᵥₑₗ*k_{gender}*k_{valence}*kₐᵣₒᵤₛₐₗ*kₒₜₕₑᵣ

The constants k of the different factors must be specifically adapted for each case in order to gain a normalized MCE result.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1a. Contributing factors to the effectiveness of marketing communication.

FIG. 1b. Brand-self perceptual neural model (BSPN).

FIG. 1c, Flowchart of evaluating effectiveness on an exposed individual.

FIG. 2a shows a composite GLM results for 15 individuals revealing a significant effect in the left and right STS and left and right Precuneus for memorable ads in comparison to unmemorable ads, the insert shows a graph of the MRI response (% BOLD signal) for memorable ads (upper curve) and unmemorable ads (lower curve);

FIG. 2b shows GLM results for 15 individuals revealing a significant effect in the left and right amygdale;

FIG. 3 shows average time course in right (upper panel) and left (lower panel) STS activity of 15 individuals, revealing consistent differences between memorable and unmemorable ads across all ads viewed (green background, memorable ads; blue background unmemorable ads);

FIG. 4a neural activation map for the STS using affect self-report measures as a predictor of memorability;

FIG. 4b neural activation map for the amygdala using affect self-report measures as a predictor of memorability;

FIG. 5 shows superimposition of the memorability and affect neural activation maps;

FIG. 6a shows the time course results for left STS for the two sessions that took place 18 months apart (upper curves-memorable ads; lower curves-unmemorable ads)

FIG. 6b shows the time course results for right STS for the two sessions that took place 18 months apart (upper curves-memorable ads; lower curves-unmemorable ads)

FIG. 7 shows a system for determining memorability of audio and visual content in accordance with one embodiment of the invention.

### DETAILED DESCRIPTION OF EMBODIMENTS

Marketing communications are messages and related media used to communicate with a market. It can also refer to the strategy used by a company or individual to reach their target market through various types of communication. Once this type of communication reaches the target market, it affects the social cognitive processes of the viewers, and induces emotional reactions.

Aspects of our current invention establish a model to evaluate the effectiveness of marketing communication, based on neural measuring of communication-elicited emotional arousal, and of social cognitive processes in the relationship with marketing communication.

Social cognition is a level of analysis that aims to understand social psychological phenomena by investigating the cognitive processes that underlie them. Social cognitive processes involve perception, judgment, and memory of social stimuli; the effects of social and affective factors on information processing; and the behavioral and interpersonal consequences of cognitive processes. All these are processes by which impressions make inferences about the self and other people. These processes make use of the attribution-contextual information cues, and are affected by differential variables such as individual's gender, motivations, emotions, cognitive load capacity, and other psychographic variables. In our brand-self perceptual neural model (BSPN) some or all of these differential variables are measured and given differential weights to determine social cognitive processing affect upon marketing communication's effectiveness.

Social cognitive processes are accompanied by emotional reactions of the communication's target audience. These reactions are commonly measured by means of emotional valence (the emotional value associated with a stimulus) and emotional arousal. Therefore our model specifically measures both valence and arousal of the viewer's reactions to the communicated stimuli, and gives these values weights in the calculation of potential marketing communication's effectiveness.

However, on top of the factors of individual's gender, valence and arousal, other differential variable factors may be considered, according to needs.

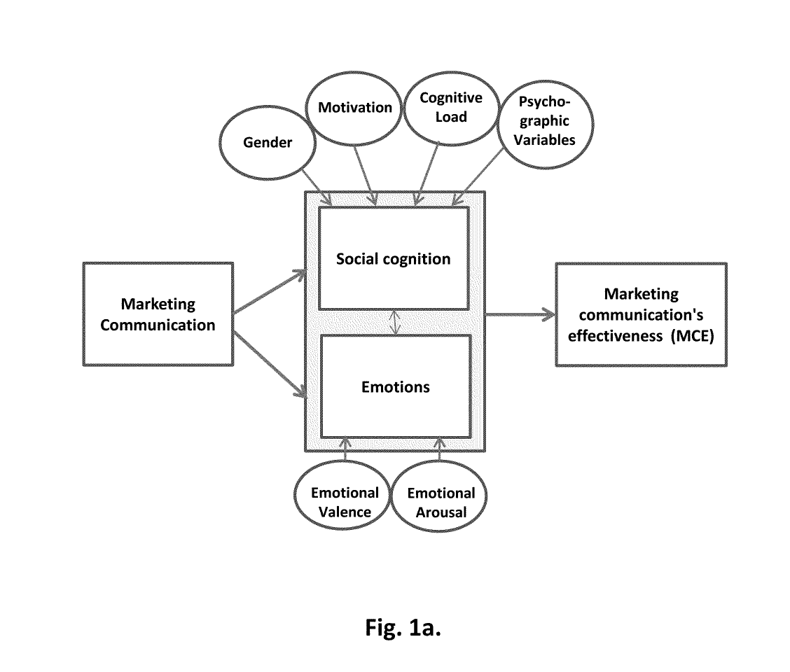

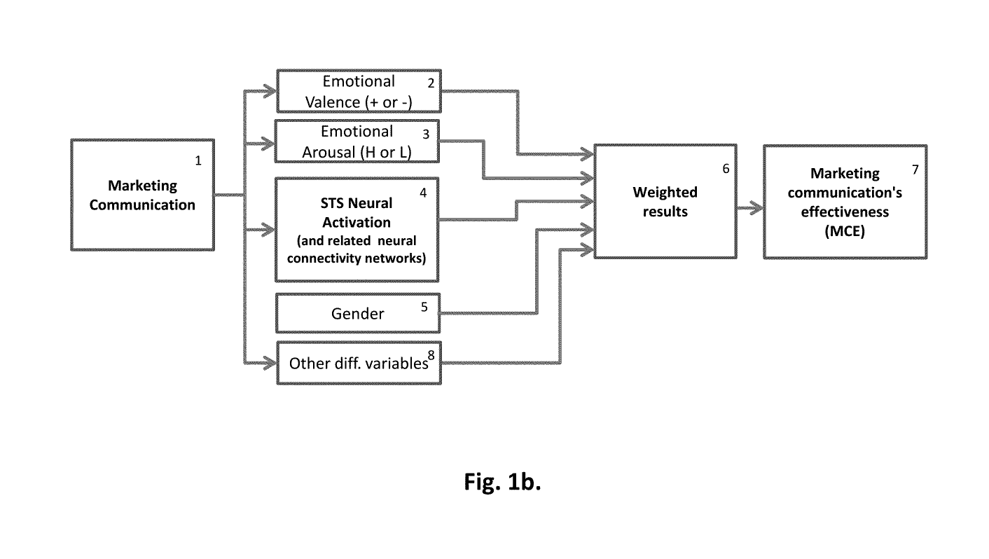

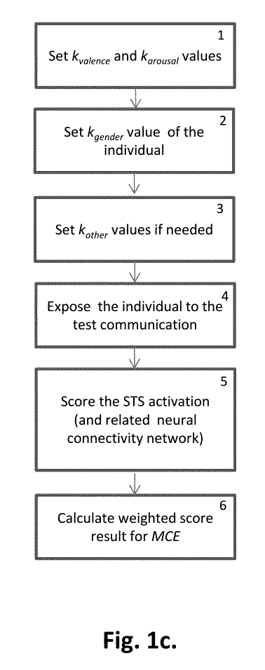

FIG. 1a depicts schematically the contributing factors to the effectiveness of market communication. Social cognition and emotion are directly indicative to the effectiveness of marketing communication. They are affected by the motivation, cognitive load and psychographic variables of the individual, and may vary according to individual's gender. The emotional valence and arousal of the marketing communication affects the degree of social cognition and emotions as well.

The underlying research to the present invention has discovered a link between ad memorability and excessive neural activation of the superior temporal sulcus (STS). The superior temporal sulcus (STS) is a brain structure indicative of social perceptual and social cognitive processes. Its activation can be measured by neural means. The proposed methodological model measures the neural activation levels and the neural patterns of the STS and related neural connectivity networks, calculates the differential effects the described above variables have upon the STS neural activation patterns (gender, motivations, emotions, cognitive load capacity, and other psychographic variables) to determine marketing communication's potential effectiveness.

FIG. 1b depicts the Brand-self perceptual neural model (BSPN) for measuring marketing communication effectiveness. A method, based on this model and practiced on a computing device, is based on neural activity measuring from the STS and the related neural connectivity networks, in the human brain. The related neural connectivity networks comprise Precuneus and Amigdale. The measured neural activation 4 is indicative of social cognition and emotions of an exposed individual to marketing material 1. Some or all of the differential variables must be weighted with the measured STS score. Emotional valence 2 is one of the differential variables having a binary value, which can invert the meaning of the individual STS score, e.g. from positive to negative attitude toward the specific marketing material. Emotional arousal 3, another differential variable, having high and low values, may indicate on a temporal emotional state of the individual induced by the marketing communication stimuli. These two parameters must be taken into account when processing the STS score (and related neural connectivity networks) for the final marketing communication effectiveness (MCE) 7. Another differential parameter is the individual's gender 5, which might be relevant in some marketing communications, but irrelevant in others. The individual's parameters of emotional valence, emotional arousal and gender, which are externally accessible, as well as other differential variables 8 according to needs, must be weighted in 6 with the STS score to normalize the final MCE result across multiple individuals. Therefore the marketing communication effectiveness (MCE) is given by the following equation:

MCE=STS_{activation}_{_{—}}ₗₑᵥₑₗ*k_{gender}*k_{valence}*kₐᵣₒᵤₛₐₗ*kₒₜₕₑᵣ

The constants k of the different factors must be specifically adapted for each case in order to gain a normalized MCE result.

As an example to which the BSPN model can be applied, let's consider an ad for cheap flights to London. Assuming that this ad addresses both genders equally, and assuming that a typical female STS score is different from that of a male, then we need to adjust the k_{gender} according to the individual's gender. We than measure emotional-valence and emotional-arousal as perceived by the stimuli's-viewers and give these parameters their weights k. Measuring emotional-valence and emotional-arousal can be done using self-report questioners filled by the neural-research's participants, or by using external representative sample.

A flowchart of the effectiveness evaluation process, as practiced on an exposed individual, is shown in FIG. 1c. The values of k_{valence} and kₐᵣₒᵤₛₐₗ are set 1. The value of k_{gender} is set 2 according to the needs of the communication matter. The value of other differential variables are set 3 as well. Then the individual is exposed to the communication stimuli (e.g. audio/visual material) 4, and the activity of the STS and related neural networks is score 5, Finally, the activity score is weighted 6 with the gender, valence and arousal constants, achieving the normalized MCE result.

As shown below, individuals, when exposed to marketing communication (messages and related media used to communicate with a market) previously determined to be memorable, showed stimulation of the STS, and the Precuneus, which was significantly greater than when exposed to ads that were previously determined to be unmemorable. The STS is a cortical structure for social cognition that governs social perception in two main domains: auditory social perception (Kriegstein and Giraud 2004) and more complex social perception, including analysis and interpretation of others (Allison, Puce, and McCarthy 2000). The Precuneus is a part of the superior parietal lobe hidden in the medial longitudinal fissure between the two cerebral hemispheres. It is sometimes described as the medial area of the superior parietal cortex.

Thus, in one of its aspects, the present invention provides a method for determining an extent of social cognitive processing which affects potential memorability of audio and/or visual content, such as marketing communication. In accordance with this aspect of the invention, one or more individuals are exposed to the audio and/or visual content, and an extent of stimulation of one or both of the Precuneus, and the STS is scored. The score is then applied to the brand-self perceptual neural model (BSPN), as one of the model's factors, for evaluating the effectiveness of marketing communication.

The scoring may be binary. In this case, if the extent of stimulation is below a predetermined threshold, a score of “0” is assigned, indicating that the content is unmemorable. If the extent of stimulation is above a predetermined threshold, a score of “1” is assigned, indicating that the content is memorable. Alternatively, a continuous scoring may be used, for example, from 0 to 1, indicative of the extent of memorability.

For example, each of one or more individuals may be presented with one or more content items, and an extent of stimulation is determined for each pair of an individual and content item. One or both of the following scoring methods could then be used:
(1) For each content item, an average and standard deviation of the determined extents of stimulation by each individual can be calculated. Content items eliciting a level of stimulation below a predetermined number of standard deviations below the average could assigned a score of “0” (unmemorable content item), while those content items eliciting a level of stimulation above a predetermined number of standard deviations above the average could be assigned a score of “1” (memorable content item).
(2) For each individual, an average and standard deviation of the determined extents of stimulation by each content element can be calculated.

In both cases, content items eliciting a level of stimulation below a predetermined number of standard deviations below the average could assigned a score of “0” (unmemorable content item), while those content items eliciting a level of stimulation above a predetermined number of standard deviations above the average could be assigned a score of “1” (memorable content item). Alternatively, a continuous scoring could be used in which each content item is assigned a score that is correlated with the number of standard deviations above or below the average of the extent of stimulation that the content element elicited.

In one embodiment, functional magnetic imaging (fMRI) is used to obtain images of one or both of the Precuneus and the STS providing indications of the level of neural stimulation in the brain. The fMRI images can then be analyzed and the extent of neural stimulation in the Precuneus and/or the STS can be scored. In other embodiments, brain imaging by other techniques, such as positron emission tomography, magnetoencephalography and single photon emission computer tomography, may be used to monitor neural activity in the Precuneus and STS.

In another of its aspects, the invention provides a system for determining memorability of audio and/or visual content. The system of the invention includes one or more devices for presenting audio and/or visual content to an individual, an apparatus for determining an extent of stimulation in one or both of the Precuneus and the STS of the individual during exposure of the individual to the audio and/or visual content, and a computing device to solve the MCE equation. For exposure of visual content, a screen may be used that is positionable in front of the individual. For exposure of audio content, loudspeakers or earphones may be used. Means for determining an extent of stimulation in one or both of the amygdale and the STS may include, for example, an fMRI apparatus, and processing means configured to analyze images obtained by the fMRI apparatus to score the neural stimulation of one or both of the amygdala and the STS.

Thus, in one of its aspects, the invention provides a system for determining marketing communication effectiveness of one or more market communication items, comprising:
(a) one or more presentation devices for presenting the marketing communication items to an individual;
(b) a monitoring apparatus for monitoring a level of neural stimulation in one or both of the Precuneus and the Superior temporal sulcus (STS) of an individual during exposure of the individual to the marketing communication items, and generating data indicative of the level of stimulation of one or both of the amygdala and the STS;
(c) a processing unit including a CPU, the CPU being configured to process data generated by the monitoring apparatus from one or more individuals to calculate one or more memorability scores of each of the one or more content items presented to the individual; and
(d) a computing device being configured to calculate the marketing communication effectiveness based on the memorability scores, emotional valence, emotional arousal and gender, applying the brand-self perceptual neural model (BSPN).

The audio and/or visual content may comprise, for example, marketing communication.

The monitoring apparatus may monitor neural stimulation only in the Precuneus, or only in the STS, or in both of the Precuneus and the STS. The monitoring apparatus may further monitor neural stimulation in the amygdale and calculation of the memorability score may further involve a level of stimulation in the amygdala.

The processing unit may further comprise a memory including one or more data files for storing data indicative of audio and/or visual content for presentation to an individual on the one or more presentation devices, and the CPU may be further configured to access the data of stored content and to present the accessed data on one or more of the presentation devices.

The monitoring apparatus may be an fMRI apparatus, in which case, the calculation of the memorability score may involve a blood oxygenation level dependent (BOLD) contrast determined by the fMRI apparatus.

The computing device may comprise any general purpose computer.

An STS neural activation scale can be built to determine memorability potential of audio and/or visual stimuli. Upon which a threshold is calculated such that stimuli above the threshold will be classified as “memorable”, and below as “unmemorable”. In order to determine the memorability potential of stimuli, this threshold is applied on a single subject neural activation level, or an average neural activation level for a group of subjects.

The system of the invention may further comprise display device, and the processing unit may be configured to display on the display device any one or more of data generated by the monitoring apparatus and scores calculated by the CPU.

In another of its aspects, the invention provides a method for determining marketing communication effectiveness of one or more marketing communication items, comprising:
a) presenting the marketing communication items to one or more individuals;
b) determining a level of neural stimulation in one or more brain regions selected from the Precuneus and the superior temporal sulcus (STS) in each of the one or more individuals during exposure of each individual to the marketing communication items, and generating data indicative of the level of stimulation of one or both of the amygdala and the STS during presentation of each of the content items; and
c) calculating one or more memorability scores of each of the one or more content items in a calculation involving the generated data.
d) measuring emotional-valence and emotional-arousal of said one or more individuals;
e) determining the gender of said one or more individuals;
f) determining other differential variables if needed
g) calculating the marketing communication effectiveness by applying the BSPN equation on the factors of memorability scores, emotional-valence, emotional-arousal, and gender for each of said individuals.

The audio and/or visual content may comprise marketing communication.

In the method of the invention, only the Precuneus may be monitored, or only the STS may be monitored. Alternatively, both the Precuneus and the STS may be monitored. The method of the invention may further comprise monitoring the amygdale in which case, calculating the memorability scores may involve a level stimulation in the amygdala.

The method may further comprise storing data indicative of audio and/or visual content for presentation to an individual on the one or more presentation devices, and accessing the data of stored content to present the accessed data on one or more devices.

The monitoring may be performed using an fMRI apparatus, in which case calculation of the memorability score may involve a blood oxygenation level dependent (BOLD) contrast determined by the fMRI apparatus.

A neural activation scale of one or more of the STS and Precueneus, can be built to determine memorability potential of audio and/or visual stimuli.

The scoring may be a binary score, wherein a score of “nonmemorable” is assigned to content generating a level of neural stimulation below a threshold and a score of “memorable” is assigned to content generating a level of stimulation above a threshold. The calculation of the score of a content item may involve calculating an average of an extent of stimulation of one or more pairs of an individual and a content item, for each of one or more content elements.

The score of a content item may assign a score that is correlated with the number of standard deviations above or below the average of the extent of stimulation that the content element elicited. The calculation of the score of a content item may involve calculating an average of an extent of stimulation of one or more pairs of an individual and a content item, for each of one or more individuals. The calculation of the score of a content item may assign a score that is correlated with the number of standard deviations above or below the average of the extent of stimulation that the content element elicited.

The measuring emotional-valence and emotional-arousal may be done using self-report questioners filled by the neural-research's participants, or by using external representative sample.

The individual's gender may be gained by individual's self-report.

The calculation of the BSPN equation may be carried on any general purpose computer.

The method may further comprise a displaying on a display device any one or more of data generated by the monitoring apparatus and scores calculated by the CPU.

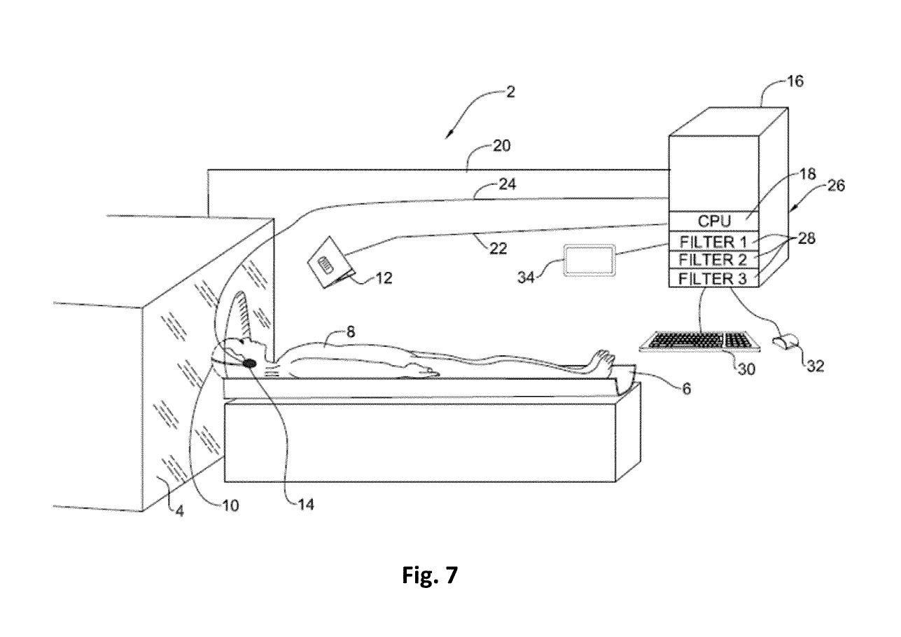

FIG. 7 shows a system 2 for determining an extent of memorability of audio and visual content, such as ads, in accordance with one embodiment of the invention. The system 2 comprises an apparatus for monitoring neural activity in one or both of the amygdala and the STS. In the embodiment of FIG. 7, the apparatus for monitoring the neural activity is an fMRI apparatus 4. A table 6 allows an individual 8 to lie with his cranium 10 (shown in phantom) inside the fMRI apparatus 4. The system 2 also comprises a screen 12 that is positioned so as to allow the individual 8 to view the screen while lying on the table 6. A pair of speakers (not shown) or a set of earphones 14 allows exposure of the individual 8 to audio content while lying on the table 6.

The system 2 further comprises a processing unit 16 that includes a CPU 18. The CPU communicates with the monitoring apparatus 4 over a communication line 20. The CPU 18 further communicates with the screen 12 over a communication line 22 and with the earphones 14 over a communication line 24. The processing unit 16 also includes a memory 26 comprising one or more files 28 where data indicative of audio and visual content may be stored prior to presenting the content to the individual 8. A user input device such as a keyboard 30 or a computer mouse 32 is used to input data into the memory, such a data identifying the subject 8 or data relating to the content to which the individual 8 is to be exposed. Processing of data provided by the monitoring apparatus is carried out by the CPU 18 and may be stored in one of the files 28 and displayed on a display device, such as a monitor 34.

The CPU 18 is configured to access content data stored in the memory 26 and to present to the individual 8 a predetermined sequence of content. The sequence of content may include, for example, one or more ads. Audio content is presented to the individual 8 by the CPU 18 over the communication line 24 to the earphones 14. Visual content is presented to the individual 8 by the CPU 18 on the screen 12 over the communication line 22. Visual and audio content may be presented simultaneously or in alternation. During presentation of the content to the individual 8, neural activity in one or both of the amygdale and the STS is monitored by the neural activity monitoring apparatus 4. Data collected by the apparatus 4 are transmitted to the processing unit 16 over the communication line 20 and are initially stored in one of the data files 28.

The CPU is configured to access the data received from the apparatus 4 and to determine a level of neural activity in one or both of the STS and the Precuneus.

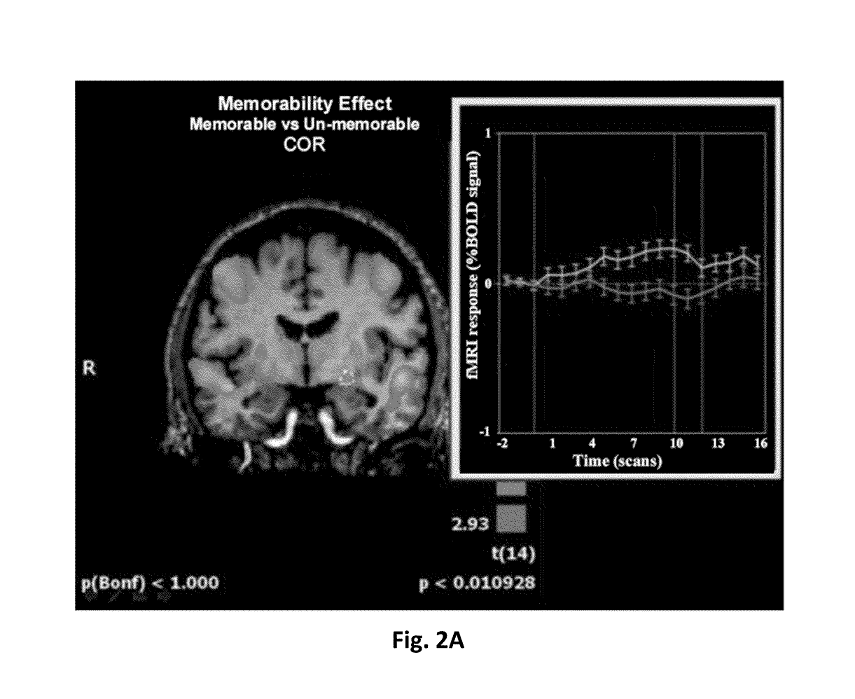

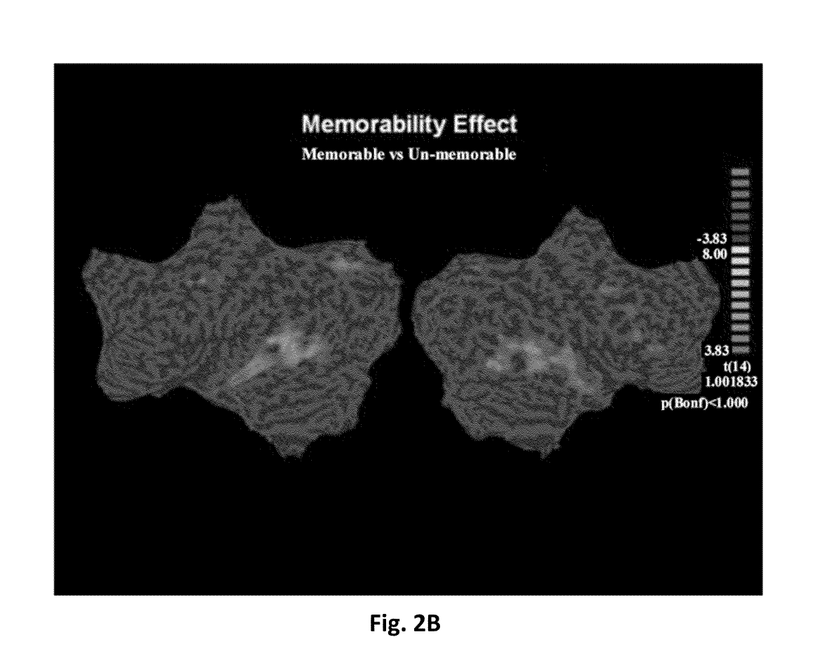

FIG. 2a shows the results of the GLM analysis in the sub-cortical structures described above for the two types of ads. The results revealed a significant effect in the left and right STS for memorable ads in comparison to unmemorable ads. The insert to FIG. 2a shows a graph of the MRI response (% BOLD signal (Kwong et al 1992)) for memorable ads (upper curve) and unmemorable ads (lower curve), which revealed significant differences in neural activation in the amygdala between memorable and unmemorable ads[q(FDR)<0.05]. FIG. 2b shows the GLM results in the cortical structures, presented on unfolded hemispheres. The results shown in FIG. 2b revealed significant differences in the overall cortical neural activations between memorable and unmemorable ads [q(FDR)<0.05]. The difference in activation between the memorable and the unmemorable stimuli was not distributed randomly across the cortex. Rather, a consistent dissimilarity in the STS was observed between memorable and unmemorable ads.

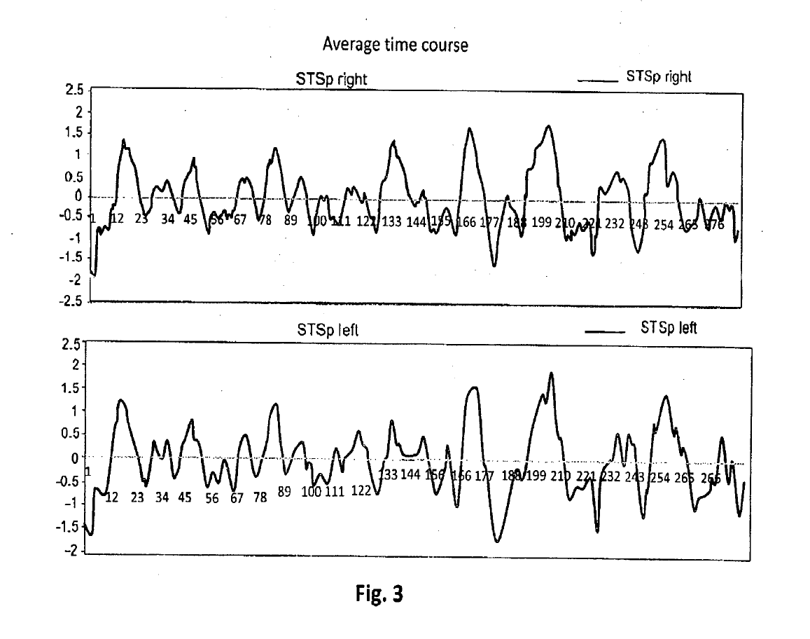

To assess whether the differences in neural activation between the memorable and unmemorable ads were consistent across every ad each of the two groups of ads, variations in BOLD activity in the STS during exposure time was studied. FIG. 3 shows the average time course in BOLD activity in right (upper panel) and left (lower panel) STS activity of the 15 individuals upon viewing each of the memorable adds (light background) and each of the unmemorable adds (dark background). The results reveal a consistently higher level of activity during exposure to ads in the memorable group compared to those in the unmemorable group.

To further explore which specific factors underlie these differences in amygdala and STS activation, the self-report measures were used including ad liking, involvement in the product, affective response, cognitive processing, and purchase intentions. To assess which of these factors is associated with memorability, each of these factors was compared across the two groups of ads. The examination revealed that the only significant measure associated with ad memorability is the affective response to the ad (t (17)=3.099, p<0.05). Analyses of all other factors revealed insignificant differences between the groups, indicating that none of the other factors (ad liking, involvement in the product, cognitive processing, or purchase intentions) can explain differences in ad memorability.

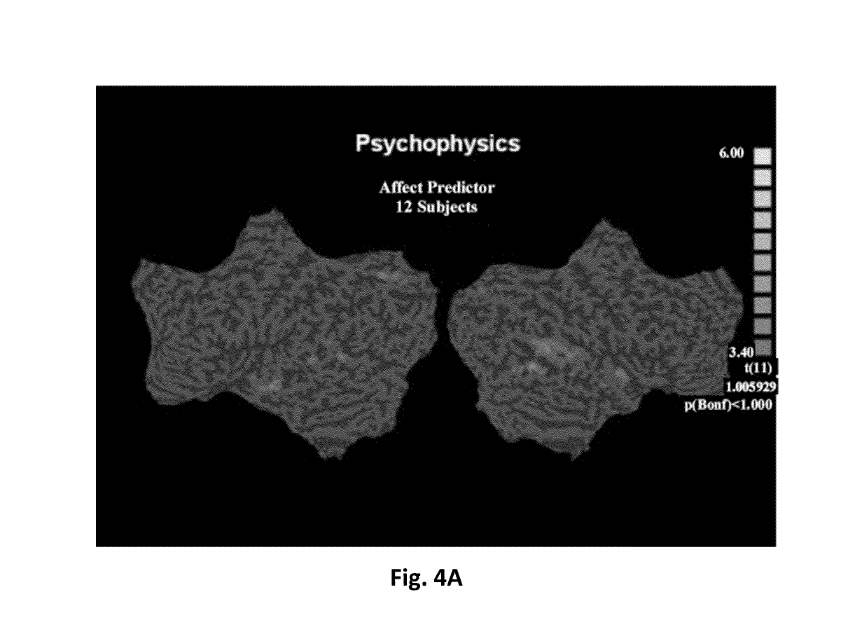

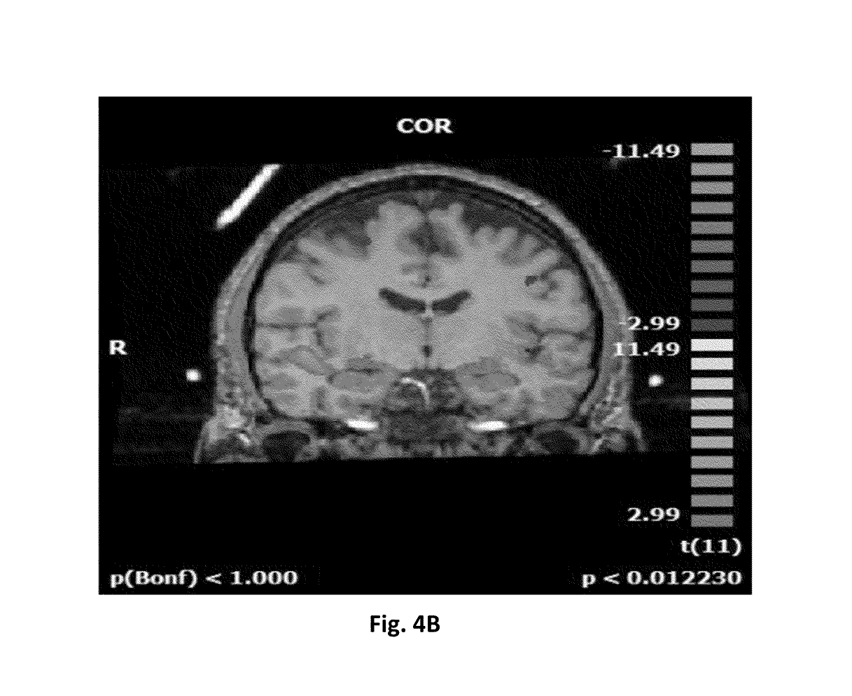

To assess whether differences in affective responses can account for the differences in amygdala and STS activation, a psychophysical multi GLM analysis was conducted by median splitting the affective response self-report measures and using it as a regressor, and the differences in the neural activation of various brain regions across the two conditions: high versus low affective response were examined. The results are shown in FIG. 4a and FIG. 4b, which show the GLM results for psychophysical analysis based on affect self-report measures as a predictor for neural activations. The results show a significant effect in the left and right amygdala (FIG. 4a) and in the STS (FIG. 4b) for affect that is consistent with the memorability effect presented above in the amygdala and STS.

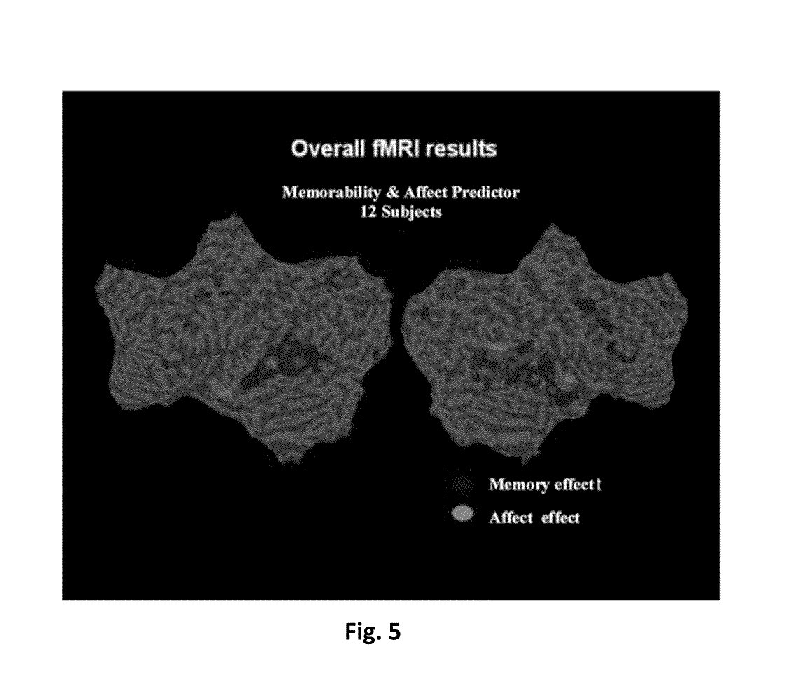

FIG. 5 shows the superimposition of the two neural maps memorability and affective effect. These results show that differences in neural activations between memorable and unmemorable ad sin the STS are associated with the affective responses the ads generated at the individual's level.

This study used real ads, and thus they could not be fully controlled for all physical characteristics. When a statistical map of the memorable versus unmemorable contrast was overlaid on the cortical mantle, no significant clusters were evident in the primary sensory cortices (see FIG. 2b, q(FDR)<0.05). This pattern of results indicates that the memory/affect effect cannot be explained by the physical low-level features of the ads.

Further examination of the stimuli reveals differences in length between the various ad groups, with a longer average time-span of the memorable compared to the unmemorable group (M_{memorable}=28.2 sec, M_{unmemorable}=20.9 sec groups; see FIG. 3). To assess whether these differences in duration have an effect on the intensity of the stimulation, the results were analyzed while controlling for ad length. The stimuli were divided into two random conditions, creating a chimera multi GLM analysis, which revealed no significant difference between two randomly selected groups of commercials [q(FDR)>0.05]. We then divided all stimuli (memorable and unmemorable) into two groups: long and short. This was done based on median splitting at 20 seconds, such that the short ads group contained nine ads ranging from 9-20 seconds, and the long ad group contained nine ads ranging from 24-39 seconds in length. A multi GLM analysis was then conducted that revealed insignificant differences between the two ad length conditions [q(FDR)>0.05]. Two separate multi GLM analyses were conducted on the two groups of ads (memorable and un-memorable), splitting each group based on ad length (i.e., memorable long vs. memorable short and un-memorable long vs. un-memorable short, with 29 seconds as the median for the memorable ads and 19 seconds as the median for the un-memorable ads). No significant differences between the two length groups were observed, neither for the memorable condition nor for the un-memorable condition [q(FDR)>0.05]. Finally, taking only those commercials balanced in length from the memorable and un-memorable commercials groups (five commercials from the memorable group and five from the un-memorable group) and an additional multi GLM analysis was performed on these smaller-scale stimuli groups. Significant differences between memorable and un-memorable commercials [q(FDR)<0.05] were found, indicating that the neural activation effect is not contingent upon ad length.

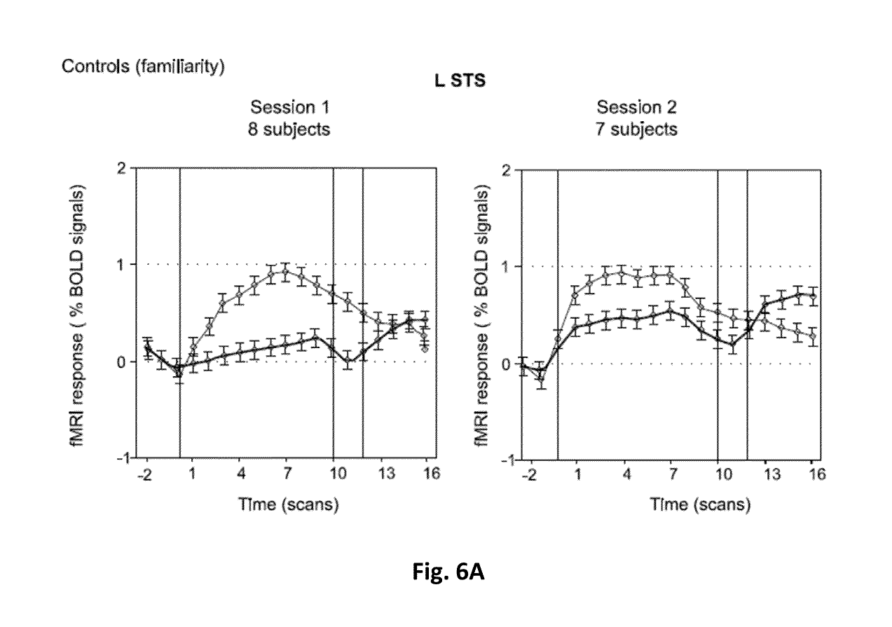

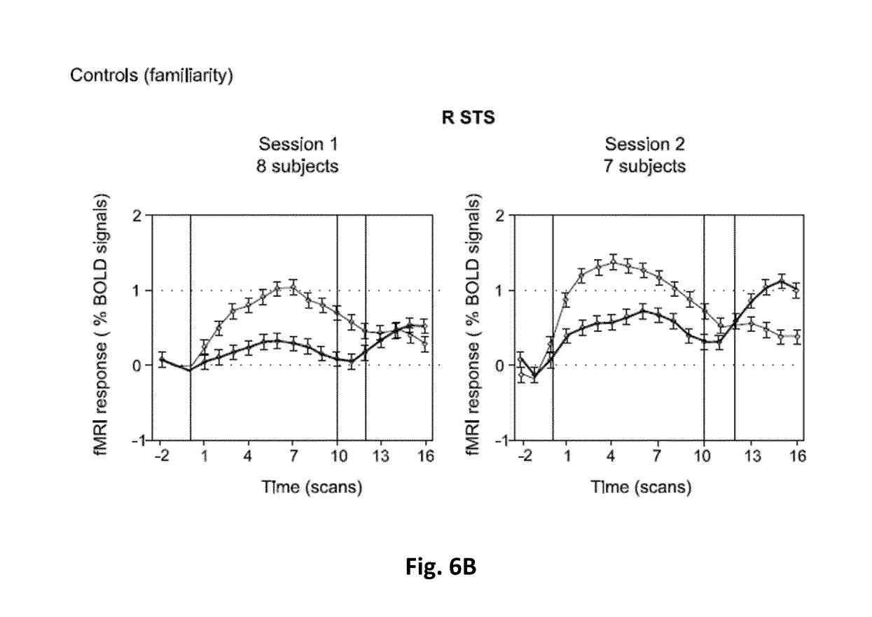

Another factor in this study was the use of real ads that were broadcasted nationally prior to the execution of the study. Thus, variations in familiarity of the individuals with the different ads might be involved in the differences observed in neural activation. The study was conducted in two sessions that took place eighteen months apart. Assuming that individuals in the first session were more familiar with the ads compared to individuals in the second session, cortical memorability effect in the two sessions were compared. This analysis revealed substantial similarities in neural activation patterns of the left STS (FIG. 6a) and the right STS (FIG. 6b) between the first and second session, suggesting that variations in familiarity of the individuals with the different ads is not a significant factor in the differences observed in neural activation.

What is claimed is:

1. A system for determining marketing communication effectiveness of one or more marketing communication items, comprising:
(a) one or more presentation devices for presenting marketing communication items to an individual;
(b) a monitoring apparatus for monitoring a level of neural stimulation in the superior temporal sulcus (STS) of an individual during exposure of the individual to the marketing communication items, and generating data indicative of the level of stimulation of the STS;
(c) a processing unit including a CPU, the CPU being configured to process data generated by the monitoring apparatus from the individual to calculate one or more memorability scores of the marketing communication items presented to the individual; and
(d) a computing device being configured to calculate the marketing communication effectiveness by applying the memorability scores and differential variables to a brand-self perceptual neural model (BSPN).

2. The system according to claim 1 wherein further the monitoring apparatus monitors neural stimulation in the Precuneus of an individual and further calculation of the memorability score by the CPU involves a level of stimulation in the Precuneus.

3. The system according to claim 2 wherein the monitoring apparatus further monitors neural stimulation in the Amygdale and calculation of the memorability score further involves a level of stimulation in the Amygdale.

4. The system according to claim 1 wherein the monitoring apparatus is an fMRI apparatus.

5. The system according to claim 1 wherein the marketing communication comprises audio and/or visual content items.

6. The system according to claim 1 wherein a score of “nonmemorable” is assigned to a marketing communication content item generating a level of neural stimulation in the STS below a calculated threshold and a score of “memorable” is assigned to an audio and/or visual content item generating a level of stimulation in the STS at or above the said threshold.

7. The system according to claim 7 wherein the said threshold is involved in calculating an extent of stimulation in the STS of one or more pairs of an individual and a content item.

8. The system according to claim 1 wherein the differential variables comprise gender, motivations, emotions, cognitive load capacity, and other psychographic variables.

9. A method for determining marketing communication effectiveness of one or more marketing communication items, comprising:
(a) presenting the marketing communication items to one or more individuals;
(b) determining a level of neural stimulation in the brain region of the superior temporal sulcus (STS) in each of the one or more individuals during exposure of each individual to the marketing communication content items, and generating data indicative of the level of stimulation of the STS during presentation of each of the content items;
(c) calculating one or more memorability scores of each of the one or more content items in a calculation involving the generated data;
(d) determining required differential variables and their values; and
(e) calculating the marketing communication effectiveness by applying the memorability scores and said differential variables to the BSPN model for each of said individuals.

10. The method according to claim 9 wherein further the Precuneus of an individual is monitored, and further calculation of the effectiveness of marketing communication involves a level of stimulation in the Precuneus.

11. The method according to claim 9 wherein further the Amigdale of an individual is monitored, and further calculation of the effectiveness of marketing communication involves a level of stimulation in the Amigdale.

12. The method according to claim 9 wherein the STS is monitored by fMRI.

13. The method according to claim 9 wherein the marketing communication comprises content of audio and/or visual items.
